(19) United States
(12) Patent Application Publication
(10) Pub. No.: US 2025/0278536 A1
(43) Pub. Date: Sep. 4, 2025

(54) MODELING METHOD FOR THERMAL RUNAWAY-ELECTROCHEMICAL COUPLING MODEL FOR CHANGE IN STATE OF CHARGE OF LITHIUM-ION BATTERY DURING CHARGING AND DISCHARGING

(71) Applicant: NANJING TECH UNIVERSITY, Nanjing (CN)

(72) Inventors: ZHIRONG WANG, Nanjing (CN); TENGFEI HE, Nanjing (CN)

(21) Appl. No.: 18/927,690

(22) Filed: Oct. 25, 2024

(30) Foreign Application Priority Data
Oct. 25, 2023 (CN) 202311399624.0

(51) Int. Cl. G06F 30/20

(52) U.S. Cl. CPC G06F 30/20

(57) ABSTRACT

The present invention relates to a modeling method for a thermal runaway-electrochemical coupling model for a change in state of charge of a lithium-ion battery during charging and discharging, and belongs to the technical field of safety of lithium-ion batteries. The method includes the following steps: S1: establishing a three-dimensional thermal runaway model of the battery under different states of charge; S21: assembling half-cells of battery cathode and anode materials; S22: testing equilibrium potentials and entropy thermal coefficients of a cathode and an anode; S23: acquiring a heat transfer coefficient between a battery surface and an ambient temperature; S24: measuring temperature and voltage change curves of the battery; S25: establishing an electrochemical model plugging electrochemical parameters into the model to obtain simulation results, and comparing the simulation results with real experimental results; and S3: making the temperatures in the electrochemical model to be consistent with an average temperature in the three-dimensional thermal runaway model under different states of charge for coupling, and setting restriction conditions after coupling. The method can achieve coupling of the thermal runaway model for the change in state of charge and electrochemistry, and can explore the thermal runaway phenomenon of batteries more comprehensively.

### TECHNICAL FIELD

The present invention belongs to the technical field of safety of lithium-ion batteries, and specifically relates to a modeling method for a thermal runaway-electrochemical coupling model for a change in state of charge of a lithium-ion battery during charging and discharging.

### BACKGROUND ART

Currently, lithium-ion batteries are widely used in electric transportation, energy storage, aerospace, etc., however, lithium-ion batteries still have serious safety issues, which has led to the limitation of the use of lithium-ion batteries.

A coupled electrochemical-thermal runaway model was developed in Journal Article 2018, Vol. 165, No. 16, A Coupled Electrochemical-Thermal Failure Model for Predicting the Thermal Runaway Behavior of Lithium-Ion Batteries by Feng, Xuning et al., which can analyze the battery voltage and the SEI membrane decomposition and reconstruction process. However, the model did not consider the change in thermal runaway characteristics caused by the change in SOC of batteries during charging and discharging.

A coupled electrochemical-thermal runaway model is developed in Journal Article 2021, Vol. 154, Modeling of thermal runaway propagation of NMC battery packs after fast charging operation by Wang, Wenhe et al. However, the model did not consider the change in thermal runaway characteristics caused by the change in SOC of batteries during charging and discharging.

A coupled electrochemical-thermal runaway model was developed in Journal Article 2018, Volume 117, Numerical modeling and analysis of the thermal behavior of NCM lithium-ion batteries subjected to very high C-rate discharge/charge operations by Dong, Ti et al., which analyzed the thermal runaway characteristics of batteries under high rate charge/discharge conditions. Similarly, the model did not consider the change in thermal runaway characteristics caused by the change in SOC of batteries during charging and discharging.

On the basis of the prior research results, the inventor has applied for the patent No. CN114864011B, entitled: Method for establishing thermal runaway three-dimensional model of lithium-ion batteries under different state of charge conditions based on differential scanning calorimeter experiments. In this patent, the experimental study shows that the thermal runaway characteristics of batteries during charging and discharging are related to the SOC of the batteries. Moreover, in this patent, control equations and boundary conditions of a thermal runaway three-dimensional model are established.

Therefore, thermal runaway models not considering the change in SOC of batteries during charging and discharging may have certain errors, and established coupled electrochemical-thermal runaway model may naturally have certain errors.

The difficulty in coupling control equations and boundary conditions of thermal runaway three-dimensional models of the existing patents with control equations and boundary conditions of electrochemistry is that it is not possible to set up a suitable coupling method and coupling conditions.

### SUMMARY OF THE INVENTION

By means of a modeling method for a thermal runaway-electrochemical coupling model for a change in state of charge of a lithium-ion battery during charging and discharging of the present invention, the problem about how to set up a suitable coupling method and coupling conditions to couple a thermal runaway model and an electrochemical model is solved.

In order to achieve the above purpose, the modeling method for the thermal runaway-electrochemical coupling model for the change in state of charge of the lithium-ion battery during charging and discharging of the present invention includes the following steps:
S1: establishing a three-dimensional thermal runaway model of the battery under different states of charge;
S2: establishing a one-dimensional electrochemical model under different ambient temperatures and discharge rates, and verifying feasibility;
S21: assembling half-cells of battery cathode and anode materials;
S22: testing equilibrium potentials and entropy thermal coefficients of a cathode and an anode in a high and low temperature test chamber and a battery test system, respectively;
S23: measuring, in the high and low temperature test chamber, a battery surface temperature curve of the battery cooled to a room temperature at a high temperature, and comparing same with simulation results to obtain a heat transfer coefficient between a battery surface and an ambient temperature;
S24: measuring, in the high and low temperature test chamber, temperature and voltage change curves of the battery under conditions of 1 C, 2 C and 3 C at ambient temperatures of 25° C., 35° C. and 45° C.; and
S25: establishing the one-dimensional electrochemical model of the battery, plugging electrochemical parameters in S22-S23 into the one-dimensional electrochemical model to obtain one-dimensional electrochemical thermal runaway simulation results, and comparing the one-dimensional electrochemical thermal runaway simulation results with real experimental results in S24 to verify the feasibility of the model; and
S3: making the temperatures in the one-dimensional electrochemical model to be consistent with an average temperature in the three-dimensional thermal runaway model under different states of charge for coupling, and setting restriction conditions after coupling.

Further, in step S3, the restriction conditions are: the coupled model conforms to an energy conservation equation:

$$ρ {C}_{p} \frac{\partial T}{\partial t} = λ {∇}^{2} T 1 + Q + q$$

By coupling in this way, the temperatures in the one-dimensional model of the battery may be consistent with the average temperature of the three-dimensional model, which may make the electrochemical reaction equation of the battery closer to the actual situation. A heat source term of the three-dimensional energy conservation equation also includes a heat source q during charging and discharging and a chemical reaction heat source Q in the thermal runaway reaction process, such that the heat source encompasses the entire reaction heat of the battery from charging and discharging to the thermal runaway process.

Heat conduction of the battery mainly considers heat conduction inside the battery and a combined heat transfer coefficient between the battery surface and the environment, i.e.:

$$- λ \frac{\partial T}{\partial n} = h ( T 1 - {T}_{a m b} ) ∘$$

where T1 denotes the battery temperature, K; h denotes the heat transfer coefficient (W/m²/K); T_{amb} denotes the ambient temperature; λ denotes the thermal conductivity of the battery material, W/m/K; and n denotes an outer normal of the heat transfer surface.

By setting the combined heat transfer coefficient in this way, the heat transfer coefficient between the battery and the environment may be measured based on experimental results, so that model results more match the experimental results.

Further, in step S3,
the SOC of the battery is defined as:
where

$$SOC = \frac{{c}_{1}}{{c}_{1 , max}} ∘ \begin{matrix} \\ {c}_{1}\end{matrix}$$

denotes the lithium concentration (mol m⁻³) in active material particles; C_{1, max} denotes the maximum concentration (mol m⁻³) of lithium in an active material; and SOC denotes the state of charge.

By defining the ratio of the maximum concentration of lithium ions in the anode of the battery and the real-time concentration of lithium ions as the SOC of the battery, the SOC of the battery may be determined in real time, and then Q values of the battery under different SOCs may be obtained.

Further, Q is defined as:

$$Q = {Q}_{total , 100 \%} \times ( 90 \% < SOC < 100 \% ) + {Q}_{total , 80 \%} \times ( 70 \% < SOC < 90 \% ) + {Q}_{total , 60 \%} \times ( 50 \% < SOC < 70 \% ) + {Q}_{total , 40 \%} \times ( 30 \% < SOC < 50 \% ) + {Q}_{total , 20 \%} \times ( 10 \% < SOC < 30 \% ) + {Q}_{total , 0 \%} \times ( 0 \% < SOC < 10 \% ) .$$

Further, in step S22, the half-cells are cycled three times at 0.2 C, and half-cells with good electrochemical performance are selected as experimental subjects; the half-cells are charged to 0%, 20%, and 40% SOC, respectively, and placed for half an hour, and open-circuit potentials of the cathode and the anode at 0%, 20%, and 40% SOC are measured, respectively; and voltages of the half-cells at 25° C., 35° C., and 45° C. SOC are measured, respectively to obtain an entropy thermal coefficient of the battery.

Similar SOCs have similar thermal runaway characteristics. In order to reduce the number of experiments under different SOCs, it is set that the thermodynamic parameters of 100% SOC are adopted when a battery is in the range of 90%-100% SOC; the thermodynamic parameters of 80% SOC are adopted when the battery is in the range of 70%-90% SOC; the thermodynamic parameters of 60% SOC are adopted when the battery is in the range of 50%-70% SOC; the thermodynamic parameters of 40% SOC are adopted when the battery is in the range of 30%-50% SOC; the thermodynamic parameters of 20% SOC are adopted when the battery is in the range of 10%-30% SOC; and the thermodynamic parameters of 0% SOC are adopted when the battery is in the range of 0%-10% SOC. By defining Q of the battery in this way, the chemical reaction heat of the battery under different SOCs may be well reflected.

### Beneficial Effects

1. The model can accurately predict the entire change in battery temperature from normal charging and discharging to thermal runaway of a lithium-ion battery during charging and discharging at different ambient temperatures, different heat transfer coefficients, and different charge and discharge rates of the battery.
2. An accurate thermal runaway model for batteries during charging and discharging can be established by testing only a small number of batteries.
3. The model can accurately predict chemical heat production inside batteries caused by temperature rise during charging and discharging.
4. The model can analyze the thermal runaway risk of the batteries under different conditions by changing the ambient temperatures, charge/discharge rates, and heat transfer coefficients of the batteries.

### BRIEF DESCRIPTION OF THE DRAWINGS

FIG. 1 shows a flowchart of a method for thermal runaway three-dimensional modeling of a change in state of charge of a lithium-ion battery during charging and discharging according to the present invention.

FIG. 2 shows battery appearance and dimensions for modeling in Embodiment 1 of the present invention.

FIG. 3 shows comparison diagrams of experimental results and simulation results under different SOC conditions in Embodiment 1 of the present invention: FIG. 3A

shows a result comparison diagram of 100% SOC; FIG. 3B shows a result comparison diagram of 80% SOC; FIG. 3C shows a result comparison diagram of 60% SOC; FIG. 3D shows a result comparison diagram of 40% SOC; FIG. 3E shows a result comparison diagram of 20% SOC; and FIG. 3F shows a result comparison diagram of 0% SOC.

FIG. 4 shows a process of determining equilibrium potentials and entropy thermal coefficients of a cathode and an anode of a battery, and a heat transfer coefficient of the battery in Embodiment 1 of the present invention: FIG. 4A shows the equilibrium potentials of the cathode and the anode; FIG. 4B shows the entropy thermal coefficients of the cathode and the anode; and FIG. 4C shows a comparison of experimental results and modeling results of the heat transfer coefficient between an outer surface of the battery and the environment.

FIG. 5 shows a comparison of experimental results and modeling results of an electro-thermal coupling model of a battery during charging and discharging in Embodiment 1 of the present invention: FIG. 5A shows a comparison of experimental results and modeling results of a battery surface temperature during discharging at an ambient temperature of 25° C.; FIG. 5B shows a comparison of experimental results and modeling results of a battery voltage during charging at the ambient temperature of 25° C.; FIG. 5C shows a comparison of experimental results and modeling results of a battery temperature during charging at different ambient temperatures; and FIG. 5D shows a comparison of experimental results and modeling results of the battery surface temperature during charging at different ambient temperatures.

FIG. 6 shows a comparison of experimental results and model results of a voltage of a battery in Embodiment 1 of the present invention during charging and discharging at ambient temperatures of 25° C., 35° C., and 45° C.; FIG. 6A shows a comparison of experimental results and modeling results of the voltage of the battery during discharging at the ambient temperature of 25° C.; FIG. 6B shows a comparison of experimental results and modeling results of the voltage of the battery during charging at the ambient temperature of 25° C.; FIG. 6C shows a comparison of experimental results and modeling results of the voltage of the battery during discharging at the ambient temperature of 35° C.; FIG. 6D shows a comparison of experimental results and modeling results of the voltage of the battery during discharging at the ambient temperature of 45° C.; FIG. 6E shows a comparison of experimental results and modeling results of the voltage of the battery during charging at the ambient temperature of 35° C.; and FIG. 6F shows a comparison of experimental results and modeling results of the voltage of the battery during charging at the ambient temperature of 45° C.

FIG. 7 shows surface temperatures and chemical reaction heat production of a battery under the conditions of different discharge rates and heat transfer coefficients (an ambient temperature of 25° C.) in Embodiment 1 of the present invention: FIG. 7A shows surface temperatures of the battery during discharging with a heat transfer coefficient of 0 W/(m²·K); FIG. 7B shows surface temperatures of the battery during discharging with a heat transfer coefficient of 1 W/(m²·K); FIG. 7C shows surface temperatures of the battery during discharging with a heat transfer coefficient of 17 W/(m²·K); FIG. 7D shows chemical reaction heat of the battery while discharging with a heat transfer coefficient of 0 W/(m²·K); FIG. 7E shows chemical reaction heat of the battery while discharging with a heat transfer coefficient of 1 W/(m²·K); and FIG. 7F shows chemical reaction heat of the battery while discharging with a heat transfer coefficient of 17 W/(m²·K).

FIG. 8 shows a process of SOC changes during discharging in Embodiment 1 of the present invention: FIG. 8A shows an SOC change of a battery when a heat transfer coefficient is 0 W/(m²·K); FIG. 8B shows an SOC change of the battery when the heat transfer coefficient is 1 W/(m²·K); and FIG. 8C shows an SOC change of the battery when the heat transfer coefficient is 17 W/(m²·K).

FIG. 9 shows graphs of a risk of thermal runaway of a battery at different ambient temperatures during discharging in Embodiment 1 of the present invention: FIG. 9A 25° C.; FIG. 9B 35° C.; and FIG. 9C 45° C.

FIG. 10 shows surface temperatures and chemical reaction heat production of a battery under the conditions of different charge rates and heat transfer coefficients (at an ambient temperature of 25° C.) in Embodiment 1 of the present invention: FIG. 10A shows a surface temperature of the battery while charging with a heat transfer coefficient of 0 W/(m²·K); FIG. 10B shows a surface temperature of the battery while charging with a heat transfer coefficient of 1 W/(m²·K); FIG. 10C shows a surface temperature of the battery while charging with a heat transfer coefficient of 17 W/(m²·K); FIG. 10D shows chemical reaction heat of the battery while charging with a heat transfer coefficient of 0 W/(m²·K); FIG. 10E shows chemical reaction heat of the battery while charging with a heat transfer coefficient of 1 W/(m²·K); and FIG. 10F shows chemical reaction heat of the battery while charging with a heat transfer coefficient of 17 W/(m²·K).

FIG. 11 shows a process of SOC changes during charging in Embodiment 1 of the present invention: FIG. 11A shows an SOC change of a battery when a heat transfer coefficient is 0 W/(m²·K); FIG. 11B shows an SOC change of the battery when the heat transfer coefficient is 1 W/(m²·K); and FIG. 11C shows an SOC change of the battery when the heat transfer coefficient is 17 W/(m²·K).

FIG. 12 shows graphs of a risk of thermal runaway of a battery at different ambient temperatures during charging in Embodiment 1 of the present invention: FIG. 12A shows a graph of the risk of thermal runaway of the battery at 25° C.; FIG. 12B shows a graph of the risk of thermal runaway of the battery at 35° C.; and FIG. 12C shows a graph of the risk of thermal runaway of the battery at 45° C.

### DETAILED DESCRIPTION OF THE INVENTION

In order to make the objective, technical solutions and advantages of embodiments of the present disclosure clearer, the technical solutions of the present invention will be clearly and completely described below with reference to the accompanying drawings. Apparently, the described embodiments are a part of the embodiments of the present invention, rather than all the embodiments. All other embodiments derived by a person of ordinary skill in the art from the embodiments of the present invention without any creative effort fall within the scope of protection of the present invention.

A modeling method for a thermal runaway-electrochemical coupling model for a change in state of charge of a lithium-ion battery during charging and discharging includes the following steps.

S1: Establish a three-dimensional thermal runaway model of the battery under different states of charge.

Step S1 is set up based on an existing patent (Publication No. CN114864011B), and the process of setting up has been indicated in the patent, including the following steps: S11: acquiring a lithium-ion battery active material with a set charge value, and conducting a differential scanning calorimeter experiment thereon to obtain a heat flow curve of the lithium-ion battery active material at different rates of temperature rising; S12: dividing the heat flow curve of the battery into a plurality of reaction peaks using a nonlinear fitting method to obtain a reaction enthalpy of each peak of the battery; S13: acquiring, based on reaction peak temperatures at the different rates of temperature rising and the reaction enthalpies of the different peaks, activation energies and pre-exponential factors of the different reaction peaks using Kissinger equation fitting; S14: fitting the heat flow curve of the battery material using genetic algorithms to obtain reaction orders and constants of the lithium-ion battery active material; S15: changing the charge value and repeating S11-S15; and S16: establishing a three-dimensional thermal runaway model.

In step S16, a control equation of the established three-dimensional thermal runaway model is as follows: the three-dimensional thermal runaway model of the battery is established based on an energy conservation equation, and the energy conservation equation when the SOC of the battery is y is shown in equation (1):

$$ρ {C}_{p} \frac{\partial T}{\partial t} = λ {∇}^{2} T 1 + {Q}_{t o t a l , y} \qquad ( 1 )$$

where t denotes the time; T₁ denotes a battery temperature (K); Cₚ denotes the specific heat capacity (J/kg/K); Q_{total, y} denotes a heat source (W/m³) when the SOC of the battery is y; λ denotes a thermal conductivity (W/m/K) of the battery material; and ρ denotes the density (kg/m³) of the battery.

The heat source term Qₜₒₜₐₗ of the battery mainly includes reaction heat between an anode of the battery and an electrolyte, reaction heat between a cathode and the anode of the battery, reaction heat of the cathode of the battery and reaction heat of a separator, as shown in equation (2):

$${Q}_{total , y} = {Q}_{caan , y} + {Q}_{sep , y} + {Q}_{a nele , y} + {Q}_{ca , y} \qquad ( 2 )$$

where Q_{caan, y} denotes the heat (W/m³) produced by the mixed material of the cathode and the anode when the SOC of the battery is y; Q_{sep, y} denotes the heat (W/m³) produced by the separator of the battery when the SOC of the battery is y; Q_{anele, y} denotes the heat (W/m³) produced by the mixed material of the anode of the battery and the electrolyte when the SOC of the battery is y; and Q_{ca, y} denotes the heat (W/m³) produced by the cathode material of the battery when the SOC of the battery is y.

The reactions in equation (2) are primarily expressed by the Areneus formula, as shown in the following equation:

$${Q}_{p} = Δ {H}_{x} \cdot {K}_{x} \cdot {W}_{x}$$

$$\frac{{dc}_{x}}{dt} = - {K}_{x} , {c}_{x , 0} = 1$$

$${K}_{x} = {A}_{x} \cdot exp ( - \frac{{E}_{a , x}}{R T 1} ) \cdot f ( {c}_{x} )$$

$$f ( {c}_{x} ) = [ {( 1 - {c}_{x} )}^{a} + p ] \cdot ( {c}_{x}^{b} )$$

where ΔHₓ denotes the reaction enthalpy (J/g) of the battery material; Kₓ denotes the rate (l/s) of decomposition reaction of the battery material; Wₓ denotes the mass fraction (kg/m³) of the battery material; Qₓ denotes the heat production (W/m³) of the battery material; Cₓ denotes the concentration of the reaction of the battery material; Aₓ denotes the pre-exponential factor of the battery material; R denotes a gas constant, 8.314 (J mol⁻²K⁻¹); E_{a, x} denotes the activation energy of the reaction peaks; and p, a, b and d denote reaction orders. The values of the above parameters other than a, b, p, and d are known, and the other parameters are shown in Tables 1-18, so the objective function is set to be Qₓ, and the variables are a, b, p, and d. The above formulas are programmed into a MATLAB program, and the Qₓ value for the best fitting objective function is obtained using the genetic algorithm to obtain the values of a, b, p, and d for the best matching Qₓ value.

In addition, the heat conduction of the battery mainly considers the heat conduction inside the battery and a combined heat transfer coefficient between the battery surface and the environment, and established boundary conditions are:

$$- λ \frac{\partial T 1}{\partial n} = h ( T 1 - {T}_{amb} )$$

where T1 denotes the battery temperature, K; h denotes the heat transfer coefficient (W/m²/K); T_{amb} denotes the ambient temperature; λ denotes the thermal conductivity of the battery material, W/m/K; and n denotes an outer normal of the heat transfer surface.

S2: Establish a one-dimensional electrochemical model of the battery.

The process includes the following steps: S21: assembling half-cells of battery cathode and anode materials, and selecting half-cells with stable capacity and performance for backup using a battery test system (Neware TS 5V10 mA);

S22: testing equilibrium potentials and entropy thermal coefficients of the battery cathode and anode materials, respectively;

S23: measuring, in a high and low temperature test chamber, a battery surface temperature of the battery cooled to a room temperature at a high temperature, and acquiring a heat transfer coefficient between the battery surface and the ambient temperature;

S24: measuring temperature and voltage change curves of the battery under charging and discharging conditions of 1 C, 2 C and 3 C at ambient temperatures of 25° C., 35° C. and 45° C., and preparing model validation of the same and temperature-voltage parameters in an electrochemical thermal coupling model; and

S25: establishing an electrochemical model.

The control equations and boundary conditions of the electrochemical model are as follows:

The electrochemical model is established mainly based on energy conservation, mass conservation, charge conservation, and electrochemical kinetics.

The charge conservation is as follows:

$$∇ \cdot ( - {σ}_{1}^{eff} ∇ {φ}_{1} ) = - {S}_{a} {j}_{l o c , 1}$$

$${S}_{a ; i} = \frac{3 {ε}_{1}}{{r}_{p}} ; {σ}_{1}^{eff} = {σ}_{1} {ε}_{1}^{{γ}_{1}}$$

$$∇ [ - {σ}_{2}^{eff} ∇ {φ}_{2} + \frac{2 R T {σ}_{2}^{eff}}{F} ( 1 + \frac{\partial ln {f}_{\pm }}{\partial ln {c}_{2}} ) ] ( 1 - {t}_{+} ) ∇ ( ln {c}_{2} ) = {S}_{a} {j}_{loc , 2}$$

$${σ}_{2}^{eff} = {σ}_{2} {ε}_{2}^{{γ}_{2}}$$

σ₁^{eff} denotes the effective solid-phase conductivity; φ₁ denotes the solid-phase potential (V); j_{loc, 1} denotes the local current density (Am⁻²) at the electrode surface; S_{a; i} denotes the specific surface area (m⁻¹); ε₁ denotes the volume fraction of the active material; rₚ denotes the radius (μm) of activated material particles; σ₁ denotes the solid-phase conductivity (S m⁻¹); σ2 denotes the liquid-phase conductivity (S m⁻¹); γ₁ denotes the Brueggemann exponent for the solid phase; γ₂ denotes the Brueggemann exponent for the liquid phase; φ₂ denotes the liquid-phase potential; σ₂^{eff} denotes the effective liquid-phase conductivity; F denotes the Faraday's constant (C mol⁻¹); ₜ₊ denotes the transport number of lithium-ion species dissolved in liquid; c₂ denotes the electrolyte concentration (mol m⁻³); and f_{±} denotes the average molar activity coefficient.

The mass conservation is shown in the following equation:

$$\frac{\partial {c}_{1}}{\partial t} + \frac{1}{{r}_{2}} \frac{\partial }{\partial r} ( - {r}^{2} {D}_{1} \frac{\partial {c}_{1}}{\partial r} ) = 0$$

$${ε}_{2} \frac{\partial {c}_{2}}{\partial t} + ∇ \cdot ( - {D}_{2}^{eff} ∇ {c}_{2} ) = \frac{{S}_{a} {j}_{l o c}}{F} ( 1 - {t}_{+} )$$

$${D}_{2}^{eff} = {D}_{2} {ε}_{2}^{{γ}_{2}}$$

where D₂^{eff} denotes the effective liquid-phase diffusion coefficient (m²s⁻¹); D₂ denotes the liquid-phase diffusion coefficient (m²s⁻¹).

The electrochemical kinetics are shown in the following equations:

$${j}_{n} = {j}_{0} [ exp ( \frac{{α}_{a ; i} F}{RT} ) η ] - exp ( - \frac{{α}_{c ; i} F}{RT} η )$$

$${j}_{0} = {Fk}_{0} {{c}_{2}^{{α}_{a}} ( {c}_{1 , max} - {c}_{1 , surf} )}^{{α}_{a}} {c}_{1 , surf}^{{α}_{a}}$$

$$η = {φ}_{1} - {φ}_{2} - {U}_{i}$$

where j₀ denotes the exchange current density (A·m⁻²); jₙ denotes the local charge transfer current density (A^{m-2}); k₀ denotes the reaction rate constant (m^{2.5}mol^{-0.5}s⁻¹); η denotes the overpotential (V), Uᵢ denotes the open-circuit voltage (V), αₐ denotes the anode current transfer coefficient, α_{c} denotes the cathode current transfer coefficient, c_{1, max} denotes the maximum lithium ion concentration (mol m⁻³), and c_{1, surf} denotes the lithium ion concentration (mol m⁻³) of the surface of the activated material particles.

The energy conservation is shown in the following equations:

$$ρ {C}_{p} \frac{\partial T}{\partial t} = k {∇}^{2} T + q$$

$$q = {q}_{rev} + {q}_{pol} + {q}_{ohm}$$

$${q}_{rev} = {S}_{a} {j}_{loc} T \frac{\partial U}{\partial T} = {S}_{a} {j}_{loc} T \frac{Δ S}{F}$$

$${q}_{pol} = {S}_{a} {j}_{loc} η$$

$${q}_{ohm} = {σ}_{1}^{eff} ∇ {φ}_{1} \cdot ∇ {φ}_{1} - [ - {σ}_{2}^{eff} ∇ {φ}_{2} + \frac{2 RT {σ}_{2}^{eff}}{F} ( 1 + \frac{\partial ln {f}_{\text{?}}}{\partial ln {c}_{2}} ) ( 1 - t \text{?} ) ∇ ( ln {c}_{2} ) ] \cdot ∇ {φ}_{2}$$

$$\text{?} \text{indicates text missing or illegible when filed}$$

where qₒₕₘ denotes the ohmic heating rate (kW·m⁻³) of the lithium-ion battery; qₚₒₗ denotes the polarization heat generation rate (kW·m⁻³) of the lithium-ion battery; qᵣₑᵥ denotes the reversible heating rate (kW·m⁻³) of the lithium-ion battery; j_{loc} denotes the local current density (Am⁻²); and ΔS denotes the entropy change.

The boundary conditions are:

$$- {σ}_{1}^{eff} \frac{\partial {φ}_{1}}{\partial x} {\vert}_{x = {L}_{n} + {L}_{s} + {L}_{p} + {L}_{n c c} + {L}_{p c c}} = - {I}_{app}$$

$${φ}_{1} {\vert}_{x = 0} = 0$$

$$- {σ}_{1}^{eff} \frac{\partial {φ}_{1}}{\partial x} {\vert}_{x = {L}_{n} + {L}_{n c c}} = - {σ}_{1}^{eff} \frac{\partial {φ}_{1}}{\partial x} {\vert}_{x = {L}_{n} + {L}_{n c c} + {L}_{s}}$$

$$\frac{\partial {φ}_{2}}{θ x} {\vert}_{x = {L}_{n c c}} = \frac{\partial {φ}_{2}}{\partial x} {\vert}_{x = {L}_{n} + {L}_{n c c} + {L}_{s} + {L}_{p}} = 0$$

$$- λ \frac{\partial T}{\partial n} = h ( T 1 - {T}_{amb} )$$

S3: Make the temperatures in the one-dimensional electrochemical model to be consistent with an average temperature in the three-dimensional thermal runaway model under different states of charge for coupling, and set restriction conditions.

T=AVGT1

AVGT1 denotes the average temperature of the three-dimensional model of the battery.

The restriction conditions include: I. The coupled model conforms to the energy conservation equation:

$$ρ {C}_{p} \frac{\partial T}{\partial t} = λ {∇}^{2} T 1 + Q + q$$

II. Heat conduction of the battery mainly considers heat conduction inside the battery and a combined heat transfer coefficient between the battery surface and the environment, i.e.

$$- λ \frac{\partial T}{\partial n} = h ( T 1 - {T}_{amb} )$$

where T1 denotes the battery temperature, K; h denotes the heat transfer coefficient (W/m²/K); T_{amb} denotes the ambient temperature; λ denotes the thermal conductivity of the battery material, W/m/K; and n denotes an outer normal of the heat transfer surface.

III. The SOC of the battery is defined as:

$$SOC = \frac{{c}_{1}}{{c}_{1 , max}}$$

where c₁ denotes the lithium concentration (mol mm⁻³) in active material particles; c_{1, max} denotes the maximum concentration (mol mm⁻³) of lithium in an active material; and SOC denotes the state of charge.

By defining the ratio of the maximum concentration of lithium ions in the anode of the battery and the real-time concentration of lithium ions as the SOC of the battery, the SOC of the battery may be determined in real time, and then Q values of the battery under different SOCs may be obtained.

IV. Q is defined as:

$$Q = {Q}_{total , 100 \%} \times ( 90 \% < SOC < 100 \% ) + {Q}_{total , 80 \%} \times ( 70 \% < SOC < 90 \% ) + {Q}_{total , 60 \%} \times ( 50 \% < SOC < 70 \% ) + {Q}_{total , 40 \%} \times ( 30 \% < SOC < 50 \% ) + {Q}_{total , 20 \%} \times ( 10 \% < SOC < 30 \% ) + {Q}_{total , 0 \%} \times ( 0 \% < SOC < 10 \% )$$

Similar SOCs have similar thermal runaway characteristics. In order to reduce the number of experiments under different SOCs, it is set that the thermodynamic parameters of 100% SOC are adopted when a battery is in the range of 90%-100% SOC; the thermodynamic parameters of 80% SOC are adopted when the battery is in the range of 70%-90% SOC; the thermodynamic parameters of 60% SOC are adopted when the battery is in the range of 50%-70% SOC; the thermodynamic parameters of 40% SOC are adopted when the battery is in the range of 30%-50% SOC; the thermodynamic parameters of 20% SOC are adopted when the battery is in the range of 10%-30% SOC; and the thermodynamic parameters of 0% SOC are adopted when the battery is in the range of 0%-10% SOC. By defining Q of the battery in this way, the chemical reaction heat of the battery under different SOCs may be well reflected.

### Embodiment 1

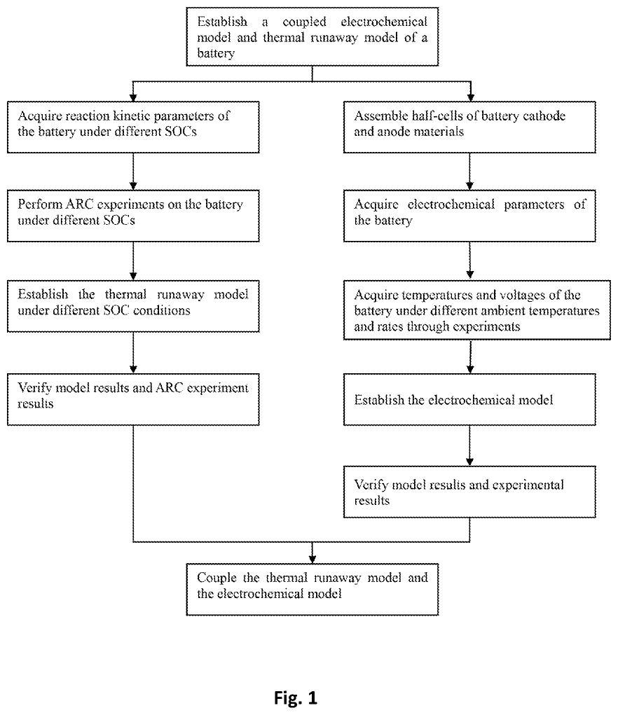

As shown in FIG. 1, a commercial 2.6 Ah 18650 type NCM523/graphite lithium-ion battery is used as an example for thermal runaway modeling of the battery and is verified with experimental results to provide a comprehensive and detailed description of the present invention. The method is not limited to this battery, but is also applicable to thermal runaway modeling of other batteries.

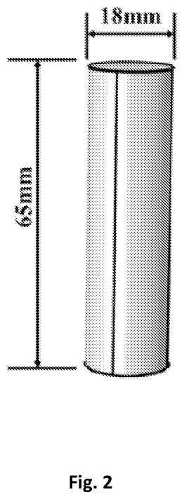

The simulation dimensions of a battery in this example are shown in FIG. 2, with a length of the battery being 65 mm and a diameter of the battery being 18 mm. The modeling mainly consists of three parts: establishment of a thermal runaway model, establishment of an electrochemical model, and coupling of the thermal runaway model and the electrochemical model.

### I. Establishment of Thermal Runaway Model

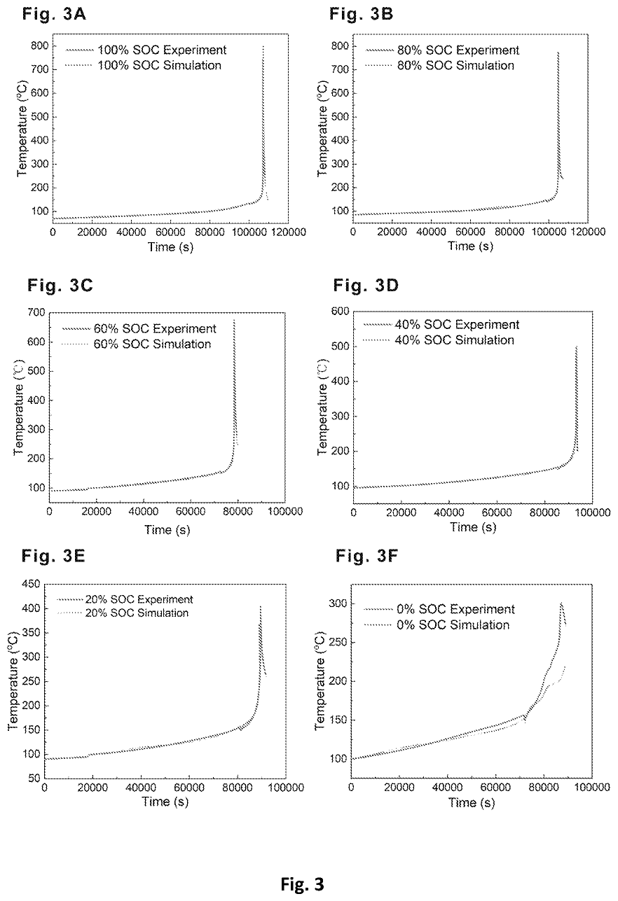

(1) First, batteries were cycled three times using a battery test system (in this example, a Neware BTS 5V6A was used) to determine the capacity and other parameters of the batteries, and a battery with good performance is selected for backup.
(2) The battery was charged to 0% SOC, 20% SOC, 40% SOC, 60% SOC, 80% SOC, and 100% SOC, respectively.
(3) Differential scanning calorimeter experiments were performed on the battery to obtain reaction kinetics parameters (i.e., battery model parameters) of the battery under different SOCs.
(4) The battery is placed into an ARC experimental apparatus for thermal runaway experiments, where a K-type thermocouple is attached to the surface of the battery to measure an actual battery temperature of the battery in a self-heating process.
(5) A three-dimensional thermal runaway model of the battery under different SOC conditions was established using COMSOL software based on the reaction kinetics parameters in (3), the parameters used in the model being shown in Tables 1-19.
(6) Model calculation results of the software in (5) were compared with the experimental results in (4) to verify the correctness of the model. The comparison results are shown in FIG. 3.

The thermal runaway reaction equations of the battery were established based on the parameters obtained from the DSC experiments, and a three-dimensional battery thermal runaway reaction model was further established based on the COMSOL software. The model parameters for 100% SOC, 80% SOC, 60% SOC,40% SOC, 20% SOC, and 0% SOC for this example are given in Tables 1-18.

TABLE 1
Battery model parameter 1 of 100% SOC

| | Caan1 | Caan2 | Caan3 | Caan4 | Caan5 | Caan6 |
|---|---|---|---|---|---|---|
| | cathode + | cathode + | cathode + | cathode + | cathode + | cathode + |
| | anode | anode | anode | anode | anode | anode |
| | reaction | reaction | reaction | reaction | reaction | reaction |
| Parameter | peak 1 | peak 2 | peak 3 | peak 4 | peak 5 | peak 6 |
| Pre-exponential | 3.98634E+26 | 1.10855E+19 | 3.02926E+14 | 29013E+13 | 821050062 | 93159283.34 |
| factor [1/s] | | | | | | |
| Reaction | 141.15 | 85.66 | 69.8 | 120.6 | 401 | 140.27 |
| enthalpy [J/g] | | | | | | |
| Activation | 2.5E+05 | 1.93E+05 | 1.66E+05 | 1.53E+05 | 1.20E+05 | 1.12E+05 |
| energy [J/mol] | | | | | | |
| Reaction | 6.1575 | 2.6349 | 0.9726 | 2.2139 | 1.2643 | 0.1932 |
| order a | | | | | | |
| Reaction | 3.4184 | 5.7262 | 0.9149 | 0 | 0.6755 | 2.5567 |
| order b | | | | | | |
| Reaction | 1.4820 | 0.5904 | 0.0303 | 0 | 0.0004 | 0.0247 |
| order p | | | | | | |
| Reaction | 169.9320 | 2.2465 | 34.1718 | 1 | 3.6017 | 14.1026 |
| order d | | | | | | |
| Active | 3.03E+02 | 3.03E+02 | 7.26E+02 | 1.51E+03 | 1.51E+03 | 1.51E+03 |
| material [kg/m³] | | | | | | |

TABLE 2
Battery model parameter 2 of 100% SOC

| | Anele1 | Anele2 | Anele3 | Anele4 |
|---|---|---|---|---|
| | electrolyte + | electrolyte + | electrolyte + | electrolyte + |
| | anode reaction | anode reaction | anode reaction | anode reaction |
| Parameter | peak 1 | peak 2 | peak 3 | peak 4 |
| Pre-exponential | 2717550891 | 147823841.2 | 28560873.82 | 7.24508E+11 |
| factor [1/s] | | | | |
| Reaction | 327.68 | 222.39 | 361.71 | 295.91 |
| enthalpy [J/g] | | | | |
| Activation | 98692.84641 | 94784.93606 | 9.05E+04 | 1.46E+05 |
| energy [J/mol] | | | | |
| Reaction | 3.8633 | 1.7547 | 1.2552 | 1.1543 |
| order a | | | | |
| Reaction | 0 | 1.3738 | 0.8535 | 1.6388 |
| order b | | | | |
| Reaction | 0 | 0.2293 | 0.0082 | 0.0209 |
| order p | | | | |
| Reaction | 1 | 0.7281 | 0.7169 | 10.3001 |
| order d | | | | |
| Active | 7.26E+02 | 7.26E+02 | 7.26E+02 | 7.26E+02 |
| material [kg/m³] | | | | |

TABLE 3
Battery model parameter 3 of 100% SOC

| | Sep Separator | Cal Cathode | Ca2 Cathode |
|---|---|---|---|
| Parameter | reaction peak | reaction peak 1 | reaction peak 2 |
| Pre-exponential | 2.0048E+44 | 5.55596E+13 | 795683294.5 |
| factor [1/s] | | | |
| Reaction | −159.51 | 20.86 | 200.1 |
| enthalpy [J/g] | | | |
| Activation | 3.48E+05 | 1.54E+05 | 1.21E+05 |
| energy [J/mol] | | | |
| Reaction order a | 1.7218 | 0.6247 | 0.1364 |
| Reaction order b | 4.2202 | 0 | 0.9546 |
| Reaction order p | 0.4471 | 0 | 0.6571 |
| Reaction order d | 0.0321 | 1 | 0.3695 |
| Active | 1.63E+02 | 6.05E+02 | 6.05E+02 |
| material [kg/m³] | | | |

TABLE 4
Battery model parameter 1 of 80% SOC

| Parameter | Caan1 | Caan2 | Caan3 | Caan4 | Caan5 |
|---|---|---|---|---|---|
| Pre-exponential | 2.45733E+28 | 1.45907E+16 | 1007495658 | 5.25869E+20 | 1.82909E+24 |
| factor [1/s] | | | | | |
| Reaction | 82 | 196.15 | 113.2 | 363.7 | 40.71 |
| enthalpy [J/g] | | | | | |
| Activation | 2.58E+05 | 1.51E+05 | 1.068E+05 | 2.42E+05 | 2.90E+05 |
| energy [J/mol] | | | | | |
| Reaction | 8.1321 | 7.2838 | 1.2056 | 1.6603 | 1.1294 |
| order a | | | | | |
| Reaction | 3.1472 | 7.5607 | 1.0501 | 0.1175 | 1.3205 |
| order b | | | | | |
| Reaction | 27.1997 | 0.0063 | 0.0037 | 0 | 0.0101 |
| order p | | | | | |
| Reaction | 27.1433 | 10.2929 | 3.5526 | 1.1358 | 10.7350 |
| order d | | | | | |
| Active | 3.03E+02 | 7.26E+02 | 1.51E+03 | 1.51E+03 | 1.51E+03 |
| material [kg/m³] | | | | | |

TABLE 5
Battery model parameter 1 of 80% SOC

| Parameter | Anele1 | Anele2 | Anele3 | Anele4 |
|---|---|---|---|---|
| Pre-exponential | 5.48776E+11 | 117056492.6 | 235178067.6 | 39791171592 |
| factor [1/s] | | | | |
| Reaction | 415.3 | 390.6 | 165.9 | 129.76 |
| enthalpy [J/g] | | | | |
| Activation | 1.1E+05 | 1.15E+05 | 1E+05 | 1.33E+05 |
| energy [J/mol] | | | | |
| Reaction | 5.5790 | 2.2918 | 1.1960 | 0.0888 |
| order a | | | | |
| Reaction | 6.6814 | 0.4887 | 1.1712 | 2.8098 |
| order b | | | | |
| Reaction | 0.0182 | 0.0018 | 0.0036 | 0.1763 |
| order p | | | | |
| Reaction | 2.4133 | 37.2361 | 1.6069 | 3.5844 |
| order d | | | | |
| Active | −7.26E+02 | 7.26E+02 | 7.26E+02 | 7.26E+02 |
| material [kg/m³] | | | | |

TABLE 6
Battery model parameter 3 of 80% SOC

| Parameter | Sep | Cal |
|---|---|---|
| Pre-exponential factor [1/s] | 2.0048E+44 | 1.158E+12 |
| Reaction enthalpy [J/g] | −159.51 | 195 |
| Activation energy [J/mol] | 3.48E+05 | 148081.6517 |
| Reaction order a | 1.7218 | 1.2343 |
| Reaction order b | 4.2202 | 1.1068 |
| Reaction order p | 0.4471 | 0.0109 |
| Reaction order d | 0.0321 | 0.9154 |
| Active material [kg/m³] | 1.63E+02 | 6.05E+02 |

TABLE 7
Battery model parameter 1 of 60% SOC

| Parameter | Caan1 | Caan2 | Caan3 | Caan4 | Caan5 |
|---|---|---|---|---|---|
| Pre-exponential | 8.68893E+22 | 3.36649E+14 | 1347317091 | 2.05353E+13 | 4.08628E+21 |
| factor [1/s] | | | | | |
| Reaction | 134.95 | 97.4 | 120.8 | 328.8 | 32.1 |
| enthalpy [J/g] | | | | | |
| Activation | 2.05E+05 | 1.4E+05 | 1.23E+05 | 1.63E+05 | 2.56E+05 |
| energy [J/mol] | | | | | |
| Reaction | 7.7895 | 3.9008 | 1.0221 | 1.2938 | 0.9718 |
| order a | | | | | |
| Reaction | 6.1035 | 6.0931 | 0.9753 | 0.5795 | 1.5671 |
| order b | | | | | |
| Reaction | 0.0252 | 0.0822 | 0.0076 | 0 | 0.0133 |
| order p | | | | | |
| Reaction | 150.1046 | 0.1587 | 42.8708 | 2.4516 | 10.2198 |
| order d | | | | | |
| Active | 3.03E+02 | 7.26E+02 | 1.51E+03 | 1.51E+03 | 1.51E+03 |
| material [kg/m³] | | | | | |

TABLE 8
Battery model parameter 2 of 60% SOC

| Parameter | Anele1 | Anele2 | Anele3 | Anele4 |
|---|---|---|---|---|
| Pre-exponential | 2.8925e+11 | 896572503.2 | 657466984.8 | 1.02692E+12 |
| factor [1/s] | | | | |
| Reaction | 328.5 | 303.5 | 313.6 | 118.4 |
| enthalpy [J/g] | | | | |
| Activation | 115600.564 | 109418.234 | 111507.368 | 146609.076 |
| energy [J/mol] | | | | |
| Reaction | 4.5229 | 1.33115 | 0.8958 | 1.7897 |
| order a | | | | |
| Reaction | 6.3980 | 0.4932268 | 0.6293 | 1.5679 |
| order b | | | | |
| Reaction | 0.0481 | 0.0000120828 | 0 | 0.0048 |
| order p | | | | |
| Reaction | 9.8520 | 1.5899 | 1.7982 | 15.7588 |
| order d | | | | |
| Active | 7.26E+02 | 7.26E+02 | 7.26E+02 | 7.26E+02 |
| material [kg/m³] | | | | |

TABLE 9
Battery model parameter 3 of 60% SOC

| Parameter | Sep | Ca1 |
|---|---|---|
| Pre-exponential factor [1/s] | 2.0048E+44 | 422708642.2 |
| Reaction enthalpy [J/g] | −159.51 | 190.86 |
| Activation energy [J/mol] | 3.48E+05 | 1.18E+05 |
| Reaction order a | 1.7218 | 0.5928 |
| Reaction order b | 4.2202 | 2.3156 |
| Reaction order p | 0.4471 | 0.0255 |
| Reaction order d | 0.0321 | 11.8629 |
| Active material [kg/m³] | 1.63E+02 | 6.05E+02 |

TABLE 10
Battery model parameter 1 of 40% SOC

| Parameter | Caan1 | Caan2 | Caan3 | Caan4 |
|---|---|---|---|---|
| Pre-exponential | 9.07847E+26 | 1.46506E+13 | 1291892633 | 7.10689E+11 |
| factor [1/s] | | | | |
| Reaction | 280 | 149.8 | 102.2 | 207 |
| enthalpy [J/g] | | | | |
| Activation | 2.7E+05 | 1.25E+05 | 1.1E+05 | 1.48E+05 |
| energy [J/mol] | | | | |
| Reaction | 16.0329 | 3.1954 | 1.0922 | 2.5800 |
| order a | | | | |
| Reaction | 182.0667 | 6.1839 | 1.2294 | 1.3511 |
| order b | | | | |
| Reaction | 454.6798 | 0.0763 | 0.0124 | 0.0104 |
| order p | | | | |
| Reaction | 454.3898 | 0.0230 | 1.7790 | 8.7991 |
| order d | | | | |
| Active | 3.03E+02 | 7.26E+02 | 1.51E+03 | 1.51E+03 |
| material [kg/m³] | | | | |

TABLE 11
Battery model parameter 2 of 40% SOC

| Parameter | Anele1 | Anele2 | Anele3 | Anele4 |
|---|---|---|---|---|
| Pre-exponential | 132338304.2 | 1066231042 | 1.07891E+11 | 949135811.5 |
| factor [1/s] | | | | |
| Reaction | 257.45 | 162.93 | 110.6 | 40.25 |
| enthalpy [J/g] | | | | |
| Activation | 99556.27607 | 109089.812 | 134736.839 | 116394.818 |
| energy [J/mol] | | | | |
| Reaction | 2.4856 | 1.2642 | 1.1735 | 0.1328 |
| order a | | | | |
| Reaction | 5.6350 | 0.7972 | 1.2043 | 4.085 |
| order b | | | | |
| Reaction | 0.3258 | 0.0059 | 0.0046 | 0.2311 |
| order p | | | | |
| Reaction | 13.0083 | 2.0219 | 8.4594 | 3.1093 |
| order d | | | | |
| Active | 7.26E+02 | 7.26E+02 | 7.26E+02 | 7.26E+02 |
| material [kg/m³] | | | | |

TABLE 12
Battery model parameter 3 of 40% SOC

| Parameter | Sep | Ca1 |
|---|---|---|
| Pre-exponential factor [1/s] | 2.0048E+44 | 1.43426E+19 |
| Reaction enthalpy [J/g] | −159.51 | 0.0453 |
| Activation energy [J/mol] | 3.48E+05 | 2.3OE+05 |
| Reaction order a | 1.7218 | 0.6338 |
| Reaction order b | 4.2202 | 1.2284 |
| Reaction order p | 0.4471 | 0.0453 |
| Reaction order d | 0.0321 | 4.5225 |
| Active material [kg/m³] | 1.63E+02 | 6.05E+02 |

TABLE 13
Battery model parameter 1 of 20% SOC

| Parameter | Caan1 | Caan2 | Caan3 | Caan4 |
|---|---|---|---|---|
| Pre-exponential | 7.10075E+16 | 86594392480 | 7.65574E+11 | 3.89456E+12 |
| factor [1/s] | | | | |
| Reaction | 79.28 | 111.9 | 116.5 | 34.7 |
| enthalpy [J/g] | | | | |
| Activation | 1.8E+05 | 1.29E+05 | 1.45E+05 | 1.59E+05 |
| energy [J/mol] | | | | |
| Reaction | 6.1059 | 3.3250 | 1.2405 | 1.2673 |
| order a | | | | |
| Reaction | 3.0613 | 5.6059 | 0.7415 | 0.7283 |
| order b | | | | |
| Reaction | 38.6689 | 0.1941 | 0.0019 | 0.0028 |
| order p | | | | |
| Reaction | 87.6900 | 15.9584 | 4.0627 | 4.2341 |
| order d | | | | |
| Active | 3.03E+02 | 7.26E+02 | 1.51E+03 | 1.51E+03 |
| material [kg/m³] | | | | |

TABLE 14
Battery model parameter 2 of 20% SOC

| Parameter | Anele1 | Anele2 | Anele3 |
|---|---|---|---|
| Pre- | 11149111061 | 7830395794 | 4.80336E+11 |
| exponential | | | |
| factor [1/s] | | | |
| Reaction | 302.7 | 153 | 92.2 |
| enthalpy | | | |
| [J/g] | | | |
| Activation | 99378.1526 | 128092.056 | 140321.7465 |
| energy | | | |
| [J/mol] | | | |
| Reaction | 3.7994 | 1.1057 | 1.1789 |
| order a | | | |
| Reaction | 3.9330 | 0.4936 | 0.8556 |
| order b | | | |
| Reaction | 0.0959 | 0.0707 | 0.0010 |
| order p | | | |
| Reaction | 0.6135 | 17.2621 | 5.6549 |
| order d | | | |
| Active | 7.26E+02 | 7.26E+02 | 7.26E+02 |
| material | | | |
| [kg/m³] | | | |

TABLE 15
Battery model parameter 3 of 20% SOC

| Parameter | Sep | Ca1 |
|---|---|---|
| Pre-exponential factor [1/s] | 2.0048E+44 | 1993236.973 |
| Reaction enthalpy [J/g] | −159.51 | 167.4 |
| Activation energy [J/mol] | 3.48E+05 | 9.23E+04 |
| Reaction order a | 1.7218 | 0.0943 |
| Reaction order b | 4.2202 | 2.8386 |
| Reaction order p | 0.4471 | 0.2174 |
| Reaction order d | 0.0321 | 3.1016 |
| Active material [kg/m³] | 1.63E+02 | 6.05E+02 |

TABLE 16
Battery model parameter 1 of 0% SOC

| Parameter | Caan1 | Caan2 | Caan3 |
|---|---|---|---|
| Pre- | 6.10595E+11 | 1.54312E+11 | 29001673.3 |
| exponential | | | |
| factor [1/s] | | | |
| Reaction | 60.3 | 30.1 | 48.5 [J/g] |
| enthalpy | | | |
| [J/g] | | | |
| Activation | 1.5E+05 | 8.5E+04 | 9.2E+04 |
| energy | | | |
| [J/mol] | | | |
| Reaction | 5.1961 | 4.3299 | 1.1415 |
| order a | | | |
| Reaction | 1.0338 | 3.3799 | 1.5705 |
| order b | | | |
| Reaction | 131.6395 | 0.0069 | 0.0174 |
| order p | | | |
| Reaction | 131.7061 | 0.0015 | 1.6094 |
| order d | | | |
| Active | 3.03E+02 | 7.26E+02 | 1.51E+03 |
| material | | | |
| [kg/m³] | | | |

TABLE 17
Battery model parameter 2 of 0% SOC

| Parameter | Anele1 | Anele2 | Anele3 |
|---|---|---|---|
| Pre- | 3684264848 | 24318583252 | 3413973065 |
| exponential | | | |
| factor [1/s] | | | |
| Reaction | 127.2 | 37.8 | 42.7 |
| enthalpy | | | |
| [J/g] | | | |
| Activation | 110939.6089 | 95433.2222 | 111199.75 |
| energy | | | |
| [J/mol] | | | |
| Reaction | 2.1098 | 1.9877 | 0.8692 |
| order a | | | |
| Reaction | 5.4766 | 1.6064 | 0.8906 |
| order b | | | |
| Reaction | 2.2879 | 0.0378 | 0.0040 |
| order p | | | |
| Reaction | 6.6854 | 0.0544 | 4.8034 |
| order d | | | |
| Active | 7.26E+02 | 7.26E+02 | 7.26E+02 |
| material | | | |
| [kg/m³] | | | |

TABLE 18
Battery model parameter 3 of 0% SOC

| Parameter | Sep | Ca1 |
|---|---|---|
| Pre-exponential factor [1/s] | 2.0048E+44 | 82557493.16 |
| Reaction enthalpy [J/g] | −159.51 | 131.2 |
| Activation energy [J/mol] | 3.48E+05 | 1.08E+05[ |
| Reaction order a | 1.7218 | 0.8631 |
| Reaction order b | 4.2202 | 1.3338 |
| Reaction order p | 0.4471 | 0.0149 |
| Reaction order d | 0.0321 | 7.9067 |
| Active material [kg/m³] | 1.63E+02 | 6.05E+02 |

### II. Establishment of Electrochemical Model

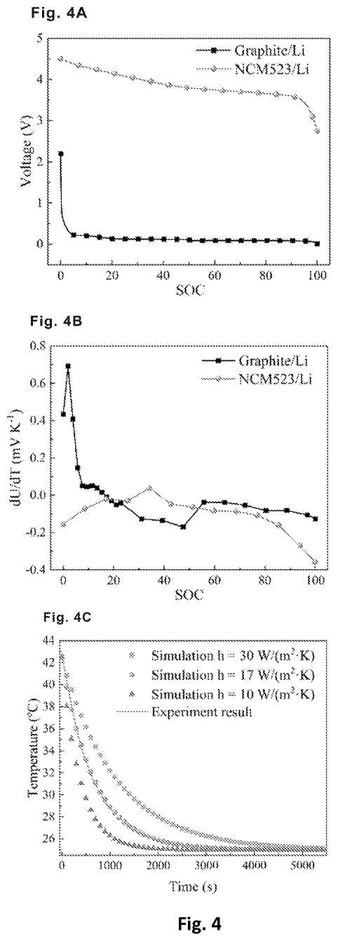

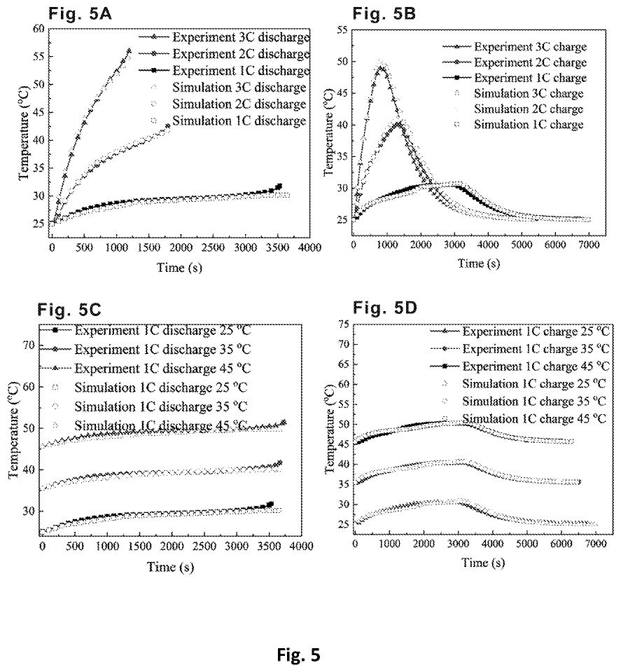

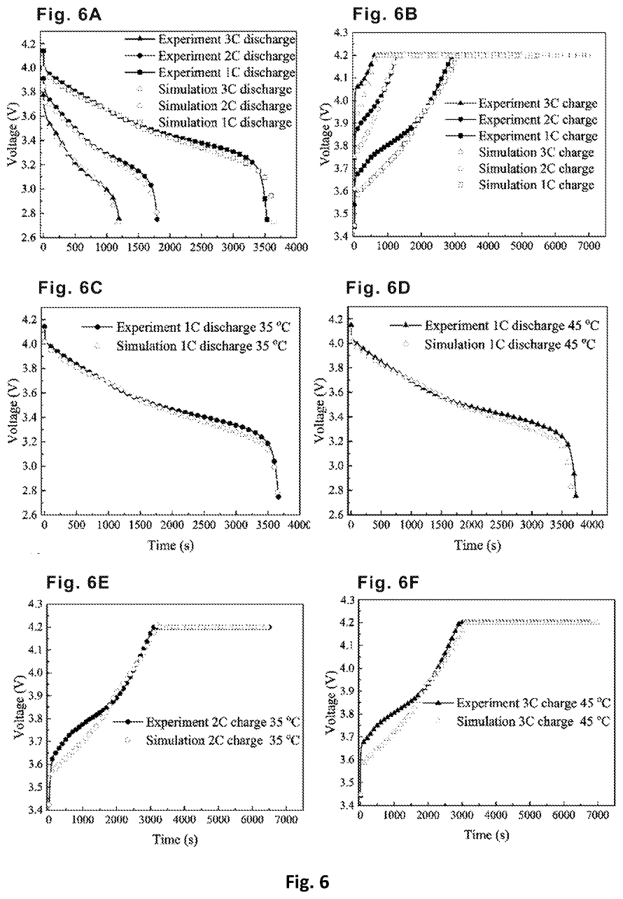

(1) NCM523/Li and graphite/Li half-cells were first assembled, and then half-cells with stable capacity and performance were selected using a battery test system (Neware BTS 5V10 mA is used in this example).
(2) The equilibrium potentials and entropy thermal coefficients of batteries are measured using a high and low temperature chamber and the battery test system. In order to obtain the equilibrium potential and entropy thermal coefficient as a function of the electrode SOC, the NCM523/Li and graphite/Li half-cells were constructed and then tested using the battery test system (Neware CT-4008-5V6A) and a low-temperature test chamber manufactured by Shanghai Yihua Climate Simulation Co., LTD. The half-cells were cycled three times at 0.2 C to determine the actual capacity, and the half-cells with good electrochemical performance were selected as experimental subjects. Then, the selected half-cells were charged to different SOCs and left for half an hour to obtain open-circuit potentials of the NCM523/Li and graphite/Li half-cells, and the equilibrium potentials at different SOCs are the open-circuit potential difference between the cathodes and anodes of the half-cells at different SOCs. In addition, voltages of the half-cells under different SOCs at different temperatures (25° C., 35° C., 45° C.) were further measured, and the entropy thermal coefficient of the batteries was obtained. The experimental results are shown in FIG. 4A and FIG. 4B.
(3) The battery surface temperature of the battery cooled to a room temperature at a high temperature was measured in the high and low temperature test chamber, and then compared with the simulation results to obtain the heat transfer coefficient (the heat transfer coefficient was 17 W/m²K) between the battery surface and the ambient temperature. The comparison results are shown in FIG. 4C.
(4) The temperature (FIG. 5) and voltage change curves (FIG. 6) of the batteries at the ambient temperatures of 25° C., 35° C., and 45° C. under 1 C, 2 C, and 3 C were measured in the high and low temperature test chamber.
(5) An electrochemical-thermal coupling model of the battery was established using COMSOL software, the model was fitted with the electrochemical parameters in (2) and (3), and comparison with the experimental results in (4) was performed to verify the correctness of the model. The comparison results are shown in FIG. 5 and FIG. 6, and the parameters used in the model are shown in Tables 19-22.

TABLE 19
Thermophysical parameters of batteries

| | | Specific | | | | Heat | |
|---|---|---|---|---|---|---|---|
| | | heat | Thermal | Thermal | Thermal | transfer | |
| | Density | capacity | conductivity | conductivity | conductivity | coefficient | Emissivity |
| Parameter | [kg/m³] | [J/kg/K] | x[W/m/K] | y[W/m/K] | z[W/m/K] | [W/m²/K] | [0] |
| Battery | 2637.63 | 1099.8 | 1.369 | 1.369 | 37.12 | 0 | 0 |

TABLE 20
Calculated cases of batteries transfer

| Ambient | Heat transfer | Discharge | Charge |
|---|---|---|---|
| temperature | coefficient | rate | rate |
| 25° C. | 0, 0.05, | 1-8 C | 1-6 C |
| | 0.2, 1, 7, 12, 17, 22, 27, | | |
| | 32, 37 Wm⁻²K⁻¹ | | |
| 35° C. | 0, 0.05, | 1-8 C | 1-6 C |
| | 0.2, 1, 7, 12, 17, 22, 27, | | |
| | 32, 37 Wm⁻²K⁻¹ | | |
| 45° C. | 0, 0.05, | 1-8 C | 1-6 C |
| | 0.2, 1, 7, 12, 17, 22, 27, | | |
| | 32, 37 Wm−²K−¹ | | |

TABLE 21
Battery model parameter 1 m²/s

| Parameter | Unit | Aluminum | Cathode | Separator | Anode | Copper |
|---|---|---|---|---|---|---|
| ε₁ | — | — | 0.43 | — | 0.384 | — |
| ε₂ | — | — | 0.4 | 0.37 | 0.444 | — |
| δ₁ | μm | 17.5 | 52 | 17.5 | 59 | 11.5 |
| r₀ | μm | — | 6 | — | 14 | — |
| c_{s, max} | mol/m³ | | 38021 | | 50507 | |
| c_{s, o, charge} | mol/m³ | | 36310 | | 14900 | |
| c_{s, o, discharge} | mol/m³ | | 8364 | | 42426 | |
| c_{2, 0} | mol/m³ | — | — | 1000 | — | — |
| αₐ | — | — | 0.5 | — | 0.5 | — |
| α_{c} | — | — | 0.5 | — | 0.5 | — |
| D_{1, ref} | m²/s | — | 1*10⁻¹³ | — | 3.9*10⁻¹⁴ | — |
| E_{aR} | kJ/mol | — | 35 | — | 20 | — |
| E_{aD} | kJ/mol | — | 25 | — | 35 | — |
| δ₁ | S/m | — | 3.8 | — | 100 | — |
| k_{0, ref} | m^{2.5}mol^{-0.5}s⁻¹ | — | 3.94*10⁻¹¹ | — | 3*10⁻¹¹ | — |
| k | — | 238 | 1.5 | 0.344 | 1.04 | 398 |
| ρ | kg m⁻³ | 1500 | 2380 | 492 | 2660 | 8900 |
| cₚ | J kg⁻¹K⁻¹ | 903 | 710 | 1978 | 1437.4 | 385 |
| T_{ref} | K | 298.15 | | | | |
| F | Cmol⁻¹ | 96487 | | | | |

Table 22
Battery model parameter 2

| | |
|---|---|
| Reaction rate constant | ${k}_{0} ( T ) = {k}_{0 , ref} exp [ \frac{{E}_{aR}}{R} ( \frac{1}{{T}_{ref}} - \frac{1}{T} ) ]$ |
| Lithium ion diffusion coefficient | ${D}_{1} ( T ) = {D}_{1 , ref} exp [ \frac{{E}_{aD}}{R} ( \frac{1}{{T}_{ref}} - \frac{1}{T} ) ]$ |
| Thermodynamic parameter | ${D}_{2} ( {c}_{2} , T ) + 1 \times {10}^{- 10} ( 3.486 + 2.809 \times {10}^{- 3} {c}_{2} - 2.798 \times {10}^{- 6} {c}_{2}^{2} + 5.297 \times 10 - {10}^{- 10} {c}_{2}^{3} ) exp [ \frac{16.5}{R} ( \frac{1}{{T}_{ref}} - \frac{1}{T} ) ]$ $v ( {c}_{2} , T ) = ( - 0.2141 + 0.001159 {c}_{2} - 7.292 \times {10}^{- 7} {c}_{2}^{2} + 1.1 \times {10}^{- 9} {c}_{2}^{3} - {3.611}^{- 13} {c}_{2}^{4} ) exp [ - \frac{1}{R} ( \frac{1}{{T}_{ref}} - \frac{1}{T} )$ |
| Ionic conductivity | ${σ}_{2} ( {c}_{2} , T ) = ( 0.002598 + 0.0002255 {c}_{2} - 1.646 \times {10}^{- 7} {c}_{2}^{2} + 3.295 \times {10}^{- 11} {c}_{2}^{3} ) exp [ \frac{4}{R} ( \frac{1}{{T}_{ref}} - \frac{1}{T} ) ]$ |
| Transport number | t₊ = 0.4038 − 0.0002438c₂ + 6.569 × 10⁻⁷ c₂² − 1.777 × 10⁻⁹c₂³ + |
| of lithium ion species | 2.005 × 10⁻¹²c₂⁴ − 9.539 × 10⁻¹⁶c₂⁵ + 1.597 × 10⁻¹⁹c₂⁶ |
| dissolved in liquid | |
| Open-circuit potential | ${U}_{eq} = {U}_{ref , i} + \frac{dU , i}{dt} ( T - {T}_{ref} )$ |
| Reaction rate constant | ${k}_{0} ( T ) = {k}_{0 , ref} exp [ \frac{{E}_{aR}}{R} ( \frac{1}{{T}_{ref}} - \frac{1}{T} ) ]$ |

### III. Coupling of Thermal Runaway Model and Electrochemical Model

The model assumes that the TR (thermal runaway) behavior of the battery is only related to the SOC during charging and discharging, and a TR chemical reaction heat source of the battery in a certain range of SOC (e.g., 90%-100% SOC) is considered to be a TR chemical reaction heat source of the battery at a specific SOC value (e.g., 100% SOC), as shown in Table 20. In addition, the temperature in the one-dimensional electrochemical model is consistent with the average temperature in the three-dimensional thermal runaway model, and accordingly the one-dimensional electrochemical model provides an electrochemical heat source for the three-dimensional thermal runaway model. Thus, establishment of the thermal runaway model of the battery due to the SOC changes of the battery during charging and discharging is ended.

### Analysis of Charging and Discharging Thermal Runaway Characteristics of Battery at Different Ambient Temperatures

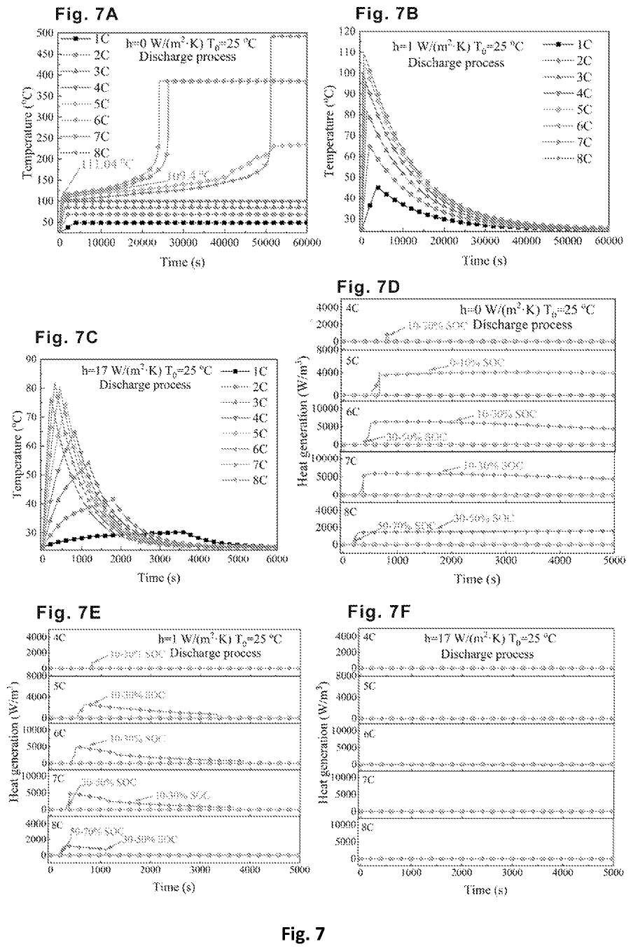

FIG. 7 shows surface temperatures and chemical reaction heat production of the battery under the conditions of different discharge rates and heat transfer coefficients (at an ambient temperature of 25° C.). Under the condition of a heat transfer coefficient of 0, it can be seen that thermal runaway of a battery occurs as the discharge rate of the battery increases. As the heat transfer coefficient increases, the surface temperature of the battery decreases during discharging and thermal runaway does not occur.

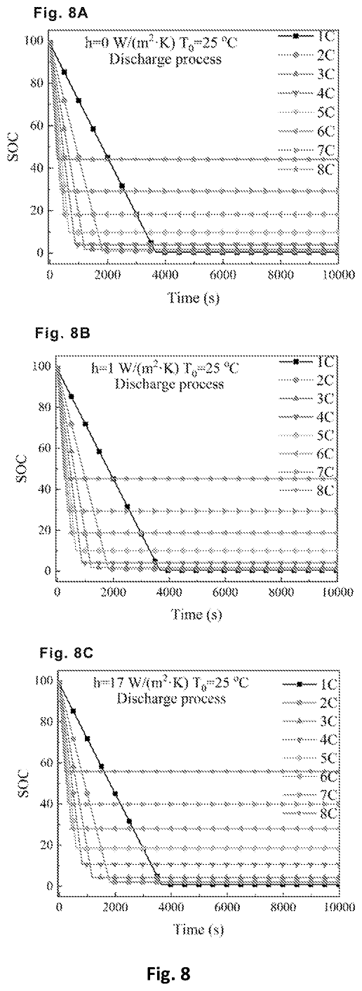

FIGS. 7D-F show chemical reaction heat production of the battery at different heat transfer coefficients and discharge rates, and the chemical reaction heat decreases as the heat transfer coefficient increases. As can be seen from FIG. 8, the SOC range in which the battery produces chemical reaction heat is higher as the discharge rate increases.

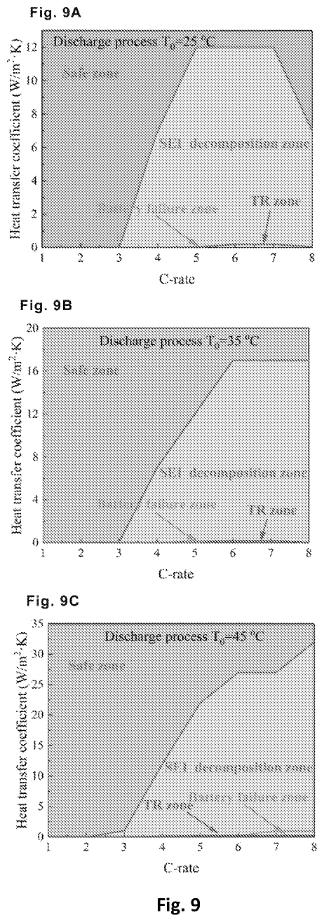

FIG. 9 shows graphs of a risk of the battery. The risk of the battery during charging and discharging is categorized into four zones:
(1) The first zone is a safe zone of the battery, in which the battery does not undergo TR and chemical reactions.
(2) The second zone is a thermal decomposition start zone. In this zone, the battery triggers chemical reactions between materials inside the battery and releases heat due to high temperatures. These chemical reactions may cause the battery to deteriorate.
(3) The third zone is a battery failure zone. When the temperature of the battery reaches 135° C., some irreversible reactions occur within the battery, which can lead to battery failure.
(4) The fourth zone is a thermal runaway zone of the battery. In this zone, the battery may trigger the thermal runaway of the battery. In the graphs of the risk, different heat transfer coefficients, ambient temperatures, and charge and discharge rates are taken as influencing conditions of the battery. This is mainly due to the fact that these three factors are common factors that will change during the use of the battery. It can be seen from FIG. 9 that the battery may easily reach a chemical reaction trigger temperature during discharge. However, the triggering of battery failure and thermal runaway is relatively difficult and may only be achieved at high discharge rates and low heat transfer coefficients. In addition, as the ambient temperature rises, the battery is more prone to chemical reaction and thermal runaway during discharging. However, as long as the heat transfer coefficient between the battery and the environment is greater than 1 W/(m²·K), battery failure or thermal runaway will not occur.

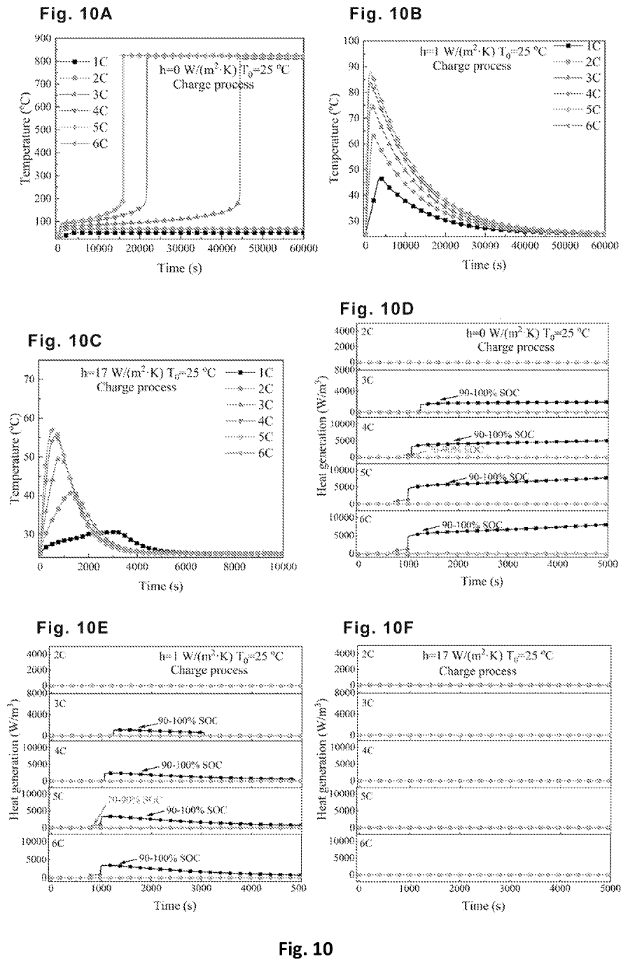

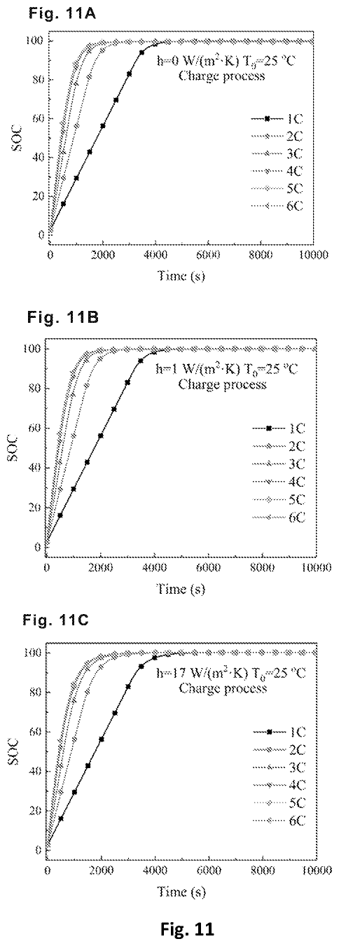

FIG. 10 shows surface temperatures and chemical reaction heat production of the battery under the conditions of different charge rates and heat transfer coefficients (at an ambient temperature of 25° C.). It can be seen from the figure that at a heat transfer coefficient of 0 W/(m²·K), the battery undergoes thermal runaway at 3 C or above. As the heat transfer coefficient increases, the temperature of the battery gradually decreases and no thermal runaway occurs. FIG. 10D-F show the chemical reaction heat production of the battery at different heat transfer coefficients and charge rates, and the chemical reaction heat decreases as the heat transfer coefficient increases. As can be seen from FIG. 11, the SOC range in which the battery produces chemical reaction heat is higher as the discharge rate increases.

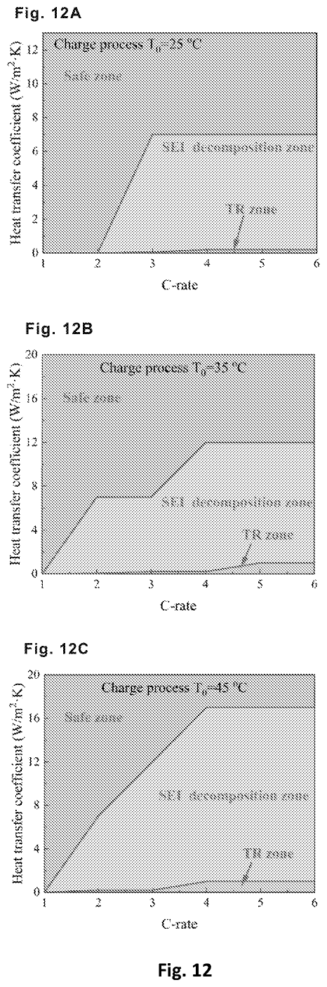

FIG. 12 shows graphs of the thermal runaway risk of the battery during charging. As shown in FIG. 12B-C, the thermal decomposition zone of the battery becomes progressively larger, indicating that higher heat transfer coefficients or lower charge rates are required to keep the battery charged in the safe zone. At all ambient temperatures, the battery failure zone and the thermal runaway zone completely overlap. This is mainly due to the fact that the SOC at the end of the battery charging process is in the range of 90-100% SOC, which generates great chemical reaction heat.

Taking the above ideal embodiment based on the present invention as a revelation, by means of the above described contents, it is entirely possible for the staff concerned to make various changes as well as modifications within the scope of not deviating from the technical ideas of the present invention. The technical scope of the present invention is not limited to the contents of the specification, but must be determined in accordance with the scope of the claims.

1. A modeling method for a thermal runaway-electrochemical coupling model for a change in state of charge of a lithium-ion battery during charging and discharging, comprising the following steps:
S1: establishing a three-dimensional thermal runaway model of the battery under different states of charge;
S2: establishing a one-dimensional electrochemical model under different ambient temperatures and discharge rates, and verifying feasibility;
S21: assembling half-cells of battery cathode and anode materials, and selecting half-cells with stable capacity and performance for backup;
S22: testing equilibrium potentials and entropy thermal coefficients of a cathode and an anode in a high and low temperature test chamber and a battery test system, respectively;
S23: measuring, in the high and low temperature test chamber, a battery surface temperature curve of the battery cooled to a room temperature at a high temperature, and comparing same with simulation results to obtain a heat transfer coefficient between a battery surface and an ambient temperature;
S24: measuring, in the high and low temperature test chamber, temperature and voltage change curves of the battery under conditions of 1 C, 2 C and 3 C at ambient temperatures of 25° C., 35° C. and 45° C.; and
S25: establishing the one-dimensional electrochemical model of the battery, plugging electrochemical parameters in S22-S23 into the one-dimensional electrochemical model to obtain one-dimensional electrochemical thermal runaway simulation results, and comparing the one-dimensional electrochemical thermal runaway simulation results with real experimental results in S24 to verify the feasibility of the model; and
S3: making the temperatures in the one-dimensional electrochemical model to be consistent with an average temperature in the three-dimensional thermal runaway model under different states of charge for coupling, and setting restriction conditions after coupling.

2. The modeling method for a thermal runaway-electrochemical coupling model for a change in state of charge of a lithium-ion battery during charging and discharging according to claim 1, characterized in that in step S3, the restriction conditions are: the coupled model conforms to an energy conservation equation:
$$ρ {C}_{p} \frac{\partial T}{\partial t} = λ {∇}^{2} T + Q + q$$
heat conduction of the battery mainly considers heat conduction inside the battery and a combined heat transfer coefficient between the battery surface and the environment, i.e:
$$- λ \frac{\partial T}{\partial n} = h ( T 1 - {T}_{a m b} ) ;$$
where T1 denotes the battery temperature, K; h denotes the heat transfer coefficient (W/m²/K); T_{amb} denotes the ambient temperature; λ denotes the thermal conductivity of the battery material, W/m/K; and n denotes an outer normal of the heat transfer surface.

3. The modeling method for a thermal runaway-electrochemical coupling model for a change in state of charge of a lithium-ion battery during charging and discharging according to claim 2, characterized in that in step S3,
the SOC of the battery is defined as:
$$SOC = \frac{{c}_{1}}{{c}_{1 , max}} ;$$
where c₁ is the lithium concentration (mol m⁻³) in active material particles; c_{1, max} denotes the maximum concentration (mol m⁻³) of lithium in an active material; and SOC denotes the state of charge.

4. The modeling method for a thermal runaway-electrochemical coupling model for a change in state of charge of a lithium-ion battery during charging and discharging according to claim 2, characterized in that in step S3,
Q is defined as:
Q=Q_{total, 100%}×(90%<SOC<100%)+Q_{total, 80%}×(70% <SOC<90%)+Q_{total, 60%}×(50%<SOC<70%)+Q_{total, 40%}×(30%<SOC<50%)+Q_{total, 20%}×(10% SOC<30%)+Q_{total, 0%}×(0%<SOC<10%).

5. The modeling method for a thermal runaway-electrochemical coupling model for a change in state of charge of a lithium-ion battery during charging and discharging according to claim 2, characterized in that in step S22, the half-cells are cycled three times at 0.2 C, and half-cells with good electrochemical performance are selected as experimental subjects; the half-cells are charged to 0%, 20%, and 40% SOC, respectively, and placed for half an hour, and open-circuit potentials of the cathode and the anode at 0%, 20%, and 40% SOC are measured, respectively; and voltages of the half-cells at 25° C., 35° C., and 45° C. SOC are measured, respectively to obtain an entropy thermal coefficient of the battery.
